(19) United States
(12) Patent Application Publication
(10) Pub. No.: US 2010/0173299 A1
(43) Pub. Date: Jul. 8, 2010

(54) MUTANT NUCLEIC ACID RELATED TO CHRONIC MYELOPROLIFERATIVE DISORDER AND METHOD OF EVALUATING CHRONIC MYELOPROLIFERATIVE DISORDER

(75) Inventors: Hiroki Yamaguchi, Bunkyo-ku (JP); Mitsuharu Inami, Bunkyo-ku (JP)

Correspondence Address: HAMRE, SCHUMANN, MUELLER & LARSON, P.C., P.O. BOX 2902, MINNEAPOLIS, MN, 55402-0902, US

(73) Assignees: ARKRAY, INC., Kyoto-shi, Kyoto (JP); NIPPON MEDICAL SCHOOL FOUNDATION, Bunkyo-ku, Tokyo (JP)

(21) Appl. No.: 12/601,738

(22) PCT Filed: Oct. 31, 2008
(86) PCT No.: PCT/JP2008/069953

§ 371 Date: Nov. 24, 2009

(30) Foreign Application Priority Data
Nov. 7, 2007 (JP) 2007-289759

(51) Int. Cl. C12Q 1/68; C07H 21/00; C07K 14/00; G01N 33/53

(52) U.S. Cl. 435/6; 536/23.1; 530/350; 435/7.1

(57) ABSTRACT

A new mutant gene related to the onset of CMPD, particularly, the new mutant gene related to the onset of CMPD in patients who develop CMPD despite of JAK2^{V617F}-negative, and an evaluation method for evaluating CMPD are provided. By detecting the following mutation in a JAK2 gene or an EPOR gene in a biological sample of human origin, the possibility of CMPD is evaluated. (a) a mutation from G at the 2116^{th} position to A in a base sequence of SEQ ID NO: 1, (b) a deletion of a nucleotide residue from the 2121^{st} to 2126^{th} position in a base sequence of SEQ ID NO: 1, (c) a mutation from C at the 1641^{st} position to G in a base sequence of SEQ ID NO: 4.

### TECHNICAL FIELD

The present invention relates to a new mutant nucleic acid related to chronic myeloproliferative disorder (CMPD) and an evaluation method of CMPD.

### BACKGROUND ART

In recent years, with respect to a variety of diseases, diagnosis by gene analysis has been tried. According to this method, for example, it can be evaluated whether the onset of the diseases is confirmed or not, and the susceptibility to the disease, by analyzing the presence or absence of a specific mutation with respect to a specific gene. Therefore, with respect to every disease, identification of a genetic mutation that shows a relationship with the onset thereof has been conducted.

With respect to chronic myeloproliferative disorder (CMPD) such as polycythemia vera (PV), essential thrombocythemia (ET), idiopathic myelofibrosis (IMF), and the like, a specific mutation in a JAK2 (Janus kinase 2) gene has been reported as a mutation related to the diseases (nonpatent documents 1 to 4). This mutation is a mutation (JAK2^{V617F}) substituting valine (V) at the 617^{th} position in JAK2 protein for phenylalanine (F). This mutation exists in a JH2 region of the JAK2 gene, and in which guanine at the 73^{rd} position in exon 12, i.e., guanine at the 2343^{rd} position in a sequence of open reading frame (ORF) with out intron, is substituted for thymine. Further, this mutation is said to cause activation of the JAK-STAT pathway, which leads to an increase in blood-forming cells, and indeed, in Western countries, it is reported that 65% to 99% of PV patients, 23% to 72% of ET patients, and 35% to 57% of IMF patients have a mutation of JAK2^{V617F}.

However, there are cases in which some patients develop CMPD, although they show JAK2^{V617F}-negative. Especially, with respect to PV patients and ET patients, for example, it is reported that clinical characteristics such as thrombotic event, hemorrhagic event, and transformation to acute leukemia are different between Japanese and Occidental, and further, the onset rate of CMPD in JAK2^{V617F}-positive patients of Japanese is lower than that of Occidental.

[Nonpatent document 1] Tefferi A. Hematology Am Soc Hematol Educ Program. 2006; 240-245.
[Nonpatent document 2] Baxter E J, Scott L M, Campbell P J, et al. Lancet. 2005; 365: 1054-1061.
[Nonpatent document 3] Kralovics R, Passamonti F, Buser A S, et al. N Engl J. Med. 2005; 352: 1779-1790.
[Nonpatent document 4] Levine R L, Wadleigh M, Cools J, et al. Cancer Cell. 2005; 7: 387-397

### DISCLOSURE OF INVENTION

Hence, the present invention is intended to provide a new mutant gene related to CMPD, especially, a new mutant gene related to CMPD in JAK2^{V617F}-negative patients, and a CMPD evaluation method that evaluates CMPD by the detection of the same.

In order to achieve the aforementioned object, a mutant nucleic acid of the present invention is composed of at least one nucleic acid selected from the group consisting of the following (i), (ii), and (iii):

(i) a nucleic acid composed of a JAK2 gene including any one of the following mutations (a) and (b) or a fragment thereof including the mutation

(a) a mutation from G at the 2116^{th} position to A in a base sequence of SEQ ID NO: 1

(b) a deletion of a nucleotide residue from the 2121^{st} to 2126^{th} position in a base sequence of SEQ ID NO: 1

(ii) a nucleic acid composed of an EPOR gene including the following mutation (c) or a fragment thereof including the mutation

(c) a mutation from C at the 1641^{st} position to G in a base sequence of SEQ ID NO: 4

(iii) a nucleic acid composed of a base sequence complementary to at least one of the nucleic acid of (i) and the nucleic acid of (ii).

A mutant polypeptide of the present invention is composed of any one of the following polypeptides (I), and (II).

(I) a polypeptide composed of JAK2 protein including any one of the following mutations (A) and (B) or a fragment thereof including the mutation

(A) a mutation from Arg at the 541^{st} position to Lys in an amino-acid sequence of SEQ ID NO: 2

(B) a deletion of Glu at the 543^{rd} position and Asp at the 544^{th} position in an amino-acid sequence of SEQ ID NO: 2

(II) a polypeptide composed of EPOR protein including the following mutation (C) or a fragment thereof including the mutation

(C) a mutation from Pro at the 502^{nd} position to Arg in an amino-acid sequence of SEQ ID NO: 5

A marker of CMPD of the present invention includes a mutant nucleic acid of the present invention or a mutant polypeptide of the present invention.

A CMPD evaluation method of the present invention is an evaluation method for evaluating the possibility of CMPD by detecting at least one mutation of a nucleic acid and a polypeptide in a biological sample. The evaluation method includes any one of the following processes (X) and (Y):

(X) a process for detecting the presence or absence of a mutant nucleic acid of the present invention in the sample

(Y) a process for detecting the presence or absence of a mutant polypeptide of the present invention in the sample

An evaluation kit of the present invention is an evaluation kit for evaluating CMPD and includes at least one of a probe which is a polynucleotide including at least one mutation selected from the group consisting of the (a), (b), and (c), or a polynucleotide composed of a base sequence complementary thereto and which can hybridize to a mutant nucleic acid of the present invention, and

a primer which is a polynucleotide including at least one mutation selected from the group consisting of the (a), (b), and (c), or a polynucleotide composed of a base sequence complementary thereto and which can hybridize to the mutant nucleic acid of the present invention. Further, the evaluation kit of the present invention includes an antibody, an antigen thereof is a mutant polypeptide of the present invention.

The inventors found, as a result of earnest study, a new mutation related to CMPD in a human JAK2 (Janus kinase 2) gene and a human EPOR (erythropoietin receptor) gene, and reached the present invention. Because a mutant nucleic acid of the present invention and a mutant polypeptide that is an expression product thereof are related to CMPD, they can be used for a marker of CMPD. Further, detection of them allows evaluation of the possibility of the onset of CMPD. Especially, because these mutations were detected from conventionally reported JAK2^{V617F}-negative CMPD patients, the evaluation of the possibility of the onset of CMPD became possible with respect to JAK2^{V617F}-negative patients whose onset of CMPD could not be evaluated by detection of JAK2^{V617F}, by detecting a mutant nucleic acid and a mutant polypeptide of the present invention. Therefore, for example, by detecting JAK2^{V617F} in combination with a mutation of the present invention, further accurate evaluation can be achieved. Accordingly, the present invention is very useful technology in a clinical medical field, and the like. In addition, a mutation in the present invention and a relationship between the mutation and diseases are found by the inventors for the first time.

### BRIEF DESCRIPTION OF DRAWINGS

FIG. 1 is a schematic diagram of a JAK2 gene and a view showing mutation sites in Example 1 of the present invention.

FIG. 2 is a schematic diagram of an EPOR gene and a view showing mutation sites in Example 1 of the present invention.

### DESCRIPTION OF THE INVENTION

In the present invention, examples of chronic myeloproliferative disorder (CMPD) include myelofibrosis with myeloid metaplasia (MMM) such as idiopathic myelofibrosis (IMF), primary myelofibrosis (PMF) and the like; erythrocytosis such as polycythemia rubra vera, polycythemia vera (PV), secondary absolute erythrocytosis, secondary absolute polycythemia, stress erythrocytosis, stresspolycythemia, and the like; polycythemia; essential thrombocythemia (ET); and hyper eosinophilic syndrome (HES); and the like.

### <Mutant Nucleic Acid and Marker>

As described above, a mutant gene of the present invention is composed of at least one nucleic acid selected from the group consisting of the following (i), (ii), and (iii):

(i) a nucleic acid composed of a JAK2 gene including any one of the following mutations (a) and (b) or a fragment thereof including the mutation

(a) a mutation from G at the 2116^{th} position to A in a base sequence of SEQ ID NO: 1

(b) a deletion of a nucleotide residue from the 2121^{st} to 2126^{th} position in a base sequence of SEQ ID NO: 1

(ii) a nucleic acid composed of an EPOR gene including the following mutation (c) or a fragment thereof including the mutation

(c) a mutation from C at the 1641^{st} position to G in a base sequence of SEQ ID NO: 4

(iii) a nucleic acid composed of a base sequence complementary to at least one of the nucleic acid of (i) and the nucleic acid of (ii).

Mutations (a), (b), and (c) of a gene newly found by the inventors are (a) a substitution mutation of a human JAK2 gene, (b) a deletion mutation of a human JAK2 gene, and (c) a substitution mutation of an EPOR gene, respectively. A base sequence of SEQ ID NO: 1 is a DNA sequence (sense strand) in a JAK2 gene and is a cDNA sequence excluding an intron. In a base sequence of SEQ ID NO: 1, a sequence in which a base t is a base u is a mature mRNA sequence in which an intron of a JAK2 gene is spliced. In a base sequence of SEQ ID NO: 1, a region from the 495^{th} to 3893^{rd} position is CDS. A base sequence of the CDS is shown in SEQ ID NO: 3. A sequence of a JAK2 gene is, for example, registered at NCBI under the accession No. NM_{—}004972. On the other hand, a base sequence of SEQ ID NO: 4 is a DNA sequence (sense strand) of an EPOR gene and is a cDNA sequence excluding an intron. In the base sequence of SEQ ID NO: 1, a sequence in which a base t is a base u is a mature mRNA sequence in which an intron of an EPOR gene is spliced. In the base sequence of SEQ ID NO: 4, a region from the 137^{th} to 1663^{rd} position is CDS. A base sequence of the CDS is shown in SEQ ID NO: 6. In SEQ ID NO: 6, a base sequence from the 1^{st} to 72^{nd} position is a coding sequence at a signaling site of EPOR protein, and a base sequence from the 73^{rd} to 1527^{th} position is a coding sequence of mature EPOR protein. A sequence of an EPOR gene is, for example, registered at NCBI under the accession No. NM_{—}000121.

When a mutant nucleic acid of the present invention is translated, Arg at the 541^{st} position in a full-length amino-acid sequence of JAK2 protein is substituted for Lys by the mutation (a), and Glu at the 543^{rd} position and Asp at the 544^{th} position in a full-length amino-acid sequence of JAK2 protein are deleted by the mutation (b). Further, when a mutant nucleic acid of the present invention is translated, Pro at the 502^{nd} position in a full-length amino-acid sequence of EPOR protein is substituted for Arg by the mutation (c). Hereinafter, the mutation (a) is also referred to as [R541K] or [JAK2^{R541K}], the mutation (b) is also referred to as [E543-D544 del] or [JAK2^{E543-D544 del}], and the mutation (c) is also referred to as [P478R] or [EPOR^{P478R}].

In (a), (b), and (c), base sequences of SEQ ID NO: 1 and SEQ ID NO: 4 are a description for specifying a new mutation site in a JAK2 gene or an EPOR gene, and a sequence of a mutant nucleic acid of the present invention is not limited to each full-length sequence. Therefore, the mutant nucleic acid of the present invention may be a full-length gene composed of a base sequence of SEQ ID NO: 1 or SEQ ID NO: 4 or a polynucleotide composed of a partial sequence thereof as long as it has any one of the mutations described above. Specific examples thereof include CDS shown in SEQ ID NO: 3 and SEQ ID NO: 6, and a polynucleotide composed of a partial sequence thereof. A fragment of (i) includes, for example, in a base sequence of SEQ ID NO: 1, a polynucleotide composed of a contiguous 5 to 10,000 base sequence including a base (A) at the 2116^{th} position, preferably a polynucleotide composed of a contiguous 8 to 1,000 base sequence, and more preferably a polynucleotide composed of a contiguous 10 to 100 base sequence. Further, a fragment of (ii) includes, for example, in a base sequence of SEQ ID NO: 4, a polynucleotide composed of a contiguous 5 to 10,000 base sequence including a deletion of a nucleotide residue from the 2121^{st} to 2126^{th} position, preferably a polynucleotide composed of a contiguous 8 to 1,000 base sequence, and more preferably a polynucleotide composed of a contiguous 10 to 100 base sequence. Furthermore, the mutant nucleic acid of the present invention may be, for example, a nucleic acid composed of an antisense strand of a JAK2 gene or a fragment thereof, or a nucleic acid composed of an antisense strand of an EPOR gene or a fragment thereof. In this instance, as shown in (iii), the mutant nucleic acid of the present invention includes a nucleic acid composed of a base sequence complementary to at least one of a nucleic acid of (i) and a nucleic acid (ii).

Further, as long as it has any one of the mutations described above, the mutant nucleic acid of the present invention may not only be a gene composed of a base sequence of SEQ ID NO:1, a gene composed of a base sequence which does not include an intron sequence showing in SEQ ID NO:3, and their fragments, a gene composed of a base sequence of SEQ ID NO:4, a gene composed of a base sequence which does not include an intron sequence shown in SEQ ID NO:6, and their fragments, but also be a gene including an intron, its fragment, or a nucleic acid composed of a base sequence complementary thereto, for example.

Furthermore, the mutant nucleic acid of the present invention is not limited to DNA, and also may be RNA, for example. When the mutant nucleic acid of the present invention is mutant RNA, for example, as described above, [t] may be [u], in a base sequences of SEQ ID NO: 1, 3, 4, or 6.

In addition, the mutant nucleic acid may be DNA or RNA that is originally contained in the sample, for example. Also, DNA may be DNA synthesized from DNA that is originally contained in the sample by a nucleic acid amplification method such as PCR and the like, or may be cDNA synthesized from RNA (total RNA, mRNA, etc.) that is originally contained in the sample by Reverse Transcription PCR (RT-PCR), for example.

Next, a CMPD marker of the present invention includes the mutant nucleic acid of the present invention. As described above, because a mutation in the mutant nucleic acid of the present invention shows relationship with the onset of CMPD, the mutant nucleic acid of the present invention can be used as a marker of CMPD. Therefore, as will be described later, for example, the onset and the possibility of the onset of CMPD can be evaluated by detecting the presence or absence of the mutant nucleic acid.

### <Mutant Polypeptide>

As described above, a mutant polypeptide of the present invention is composed of any one of the following polypeptides (I), and (II). This mutant polypeptide is, for example, an expression product of the mutant nucleic acid of the present invention.

(I) a polypeptide composed of JAK2 protein including any one of the following mutations (A) and (B) or a fragment thereof including the mutation

(A) a mutation from Arg at the 541^{st} position to Lys in an amino-acid sequence of SEQ ID NO: 2

(B) a deletion of Glu at the 543^{rd} position and Asp at the 544^{th} position in an amino-acid sequence of SEQ ID NO: 2

(II) a polypeptide composed of EPOR protein including the following mutation (C) or a fragment thereof including the mutation

(C) a mutation from Pro at the 502^{nd} position to Arg in an amino-acid sequence of SEQ ID NO: 5

Mutations (A), (B), and (C) of a polypeptide, newly found by the inventors are (A) a substitution mutation of human JAK2 protein, (B) a deletion mutation of human JAK2 protein, and (C) a substitution mutation of EPOR protein, respectively. The amino-acid sequence of SEQ ID NO: 2 is an amino-acid sequence of JAK2 protein, and this sequence is, for example, registered at NCBI under the accession No. NM_{—}004972. The amino-acid sequence of SEQ ID NO: 5 is an amino-acid sequence of EPOR protein, and the sequence is, for example, registered at NCBI under the accession No. NM_{—}000121. In the SEQ ID NO: 5, an amino-acid sequence from the 1St to 24^{th} position is a signal sequence of EPOR protein, and an amino-acid sequence from the 25^{th} to 508^{th} position is a sequence of mature EPOR protein excluding a signal sequence.

In (A), (B), and (C), amino-acid sequences of SEQ ID NO: 2 and SEQ ID NO: 5 are a description for specifying a mutation site, and a sequence of the mutant polypeptide of the present invention is not limited to each full-length sequence. Therefore, as long as it has any one of the mutations described above, the mutant polypeptide of the present invention may be a full-length polypeptide (protein) composed of an amino-acid sequence of SEQ ID NO: 2 or SEQ ID NO: 5 or may be a polypeptide fragment composed of a partial sequence thereof. Specific examples thereof include mature EPOR protein from the 25^{th} to 508^{th} position of SEQ ID NO: 5 and a polypeptide fragment composed of a partial sequence thereof.

Next, a CMPD marker of the present invention includes the mutant polypeptide of the present invention. As described above, because the mutation in the mutant polypeptide of the present invention shows a relationship with the onset of CMPD, the mutant polypeptide of the present invention can be used as a marker of CMPD. Therefore, as will be described later, the onset and the possibility of the onset of CMPD can be evaluated by detecting the presence or absence of the mutant polypeptide.

### <CMPD Evaluation Method>

A CMPD evaluation method of the present invention is an evaluation method for evaluating the possibility of CMPD by detecting at least one mutation of a nucleic acid and a polypeptide in a biological sample. The evaluation method includes at least one of the following processes (X) and (Y):

(X) a process for detecting the presence or absence of a mutant nucleic acid of the present invention in the sample

(Y) a process for detecting the presence or absence of a mutant polypeptide of the present invention in the sample

In the present invention, any one of the mutant nucleic acid and the mutant polypeptide of the present invention may be detected. When the mutant nucleic acid or the mutant polypeptide of the present invention is detected, for example, it may be judged as CMPD or susceptibility to CMPD. When the mutant nucleic acid or the mutant polypeptide of the present invention is not detected, for example, it may be judged as having low susceptibility to CMPD or no susceptibility to CMPD. In addition, the method for detecting the mutant nucleic acid of the present invention and the method for detecting the mutant polypeptide of the present invention are not particularly limited and the conventionally known method of detecting a mutation can be adopted.

Examples of a sample to be subjected to the present invention include a hemocyte sample such as a leukocyte cell and the like; a whole blood sample; a bone-marrow sample; and the like, although it is not particularly limited. Further, the sample is, for example, preferably a biological sample of human or a biological sample of mammals excluding human.

The processes (X) and (Y) respectively are described below. However, they are mere examples and do not limit the present invention.

### Embodiment 1

As the first embodiment, an example of the process (X), i.e., a process of detecting the presence or absence of the mutant nucleic acid of the present invention in the sample is described.

The presence or absence of a mutant nucleic acid in the sample can be detected by detecting at least one mutation selected from the group consisting of the following (a), (b), and (c) with respect to the nucleic acid in the sample, for example.

(a) a mutation from G at the 2116^{th} position to A in a base sequence of SEQ ID NO: 1

(b) a deletion of a nucleotide residue from the 2121^{st} to 2126^{th} position in a base sequence of SEQ ID NO: 1

(c) a mutation from C at the 1641^{st} position to G in a base sequence of SEQ ID NO: 4

The detection of the mutation may be conducted with respect to any one of them, two or more of them, or all of them. Further, the mutations of the (a) and (b) preferably are detected together.

The mutation can be detected, by hybridizing a nucleic acid in the sample with a probe that can hybridize specifically to a region including the mutation, for example. In this instance, when the hybridization between the probe and the nucleic acid in the sample is confirmed, it can be judged as having a mutation, and when the hybridization is not confirmed, it can be judged as not having a mutation. An example of the probe includes a polynucleotide including any one of the mutations in the (a), (b), and (c) or a polynucleotide composed of a base sequence complementary thereto, and which can hybridize to a mutant nucleic acid including the mutation. Examples of this kind of detection method include an Invader method, a Tm analysis method, and the like.

Further, the mutation can be detected by, for example, conducting a nucleic acid amplification reaction in which a nucleic acid in the sample is used as a template with a primer that can hybridize specifically to a region including the mutation. In this instance, when an amplification product is obtained by a nucleic acid amplification reaction, it can be judged as having a mutation, and when an amplification product is not obtained, it can be judged as not having a mutation. An example of the primer includes a polynucleotide including at least one of the mutations selected from the group (a), (b), and (c) or a polynucleotide composed of a base sequence complementary thereto, and which can hybridize to the mutant nucleic acid including the mutation. Examples of this kind of detection method include an ASP (allele specific primer)—PCR method, and the like. In this instance, as the primer, preferably a primer which has the mutant base or a base complementary to the mutant base at the 3′ region, especially at the 3′ end or the first base or the second base from the 3′ end is used.

In addition, examples of the method include a Direct Sequencing method, a Pyrosequencing method, a Denaturing HPLC method, and the like. In the Direct Sequencing method and the Pyrosequencing method, with respect to a nucleic acid in the sample, a region including a site where a target mutation occurs is amplified and the entire base sequence of an amplification product is analyzed, and in the Denaturing HPLC method, HPLC is performed with respect to the amplification product in a temperature gradient column and the presence or absence of a mutation is detected from an elution time.

The nucleic acid amplification method is not limited at all, and examples thereof include a PCR method, a Nucleic acid sequence based amplification (NASBA) method, a Transcription-mediated amplification (TMA) method, a Strand Displacement Amplification (SDA) method, and the like. In addition, the conditions of a nucleic acid amplification reaction are not particularly limited and can be conducted by the conventionally known method.

### Embodiment 2

As the second embodiment, an example of the process (Y), i.e., a process of detecting the presence or absence of a mutant polypeptide of the present invention in the sample is described.

The presence or absence of a mutant polypeptide in the sample can be detected by detecting at least one mutation selected from the group consisting of the following (A), (B), and (C) with respect to a polypeptide in the sample, for example.

(A) a mutation from Arg at the 541^{st} position to Lys in an amino-acid sequence of SEQ ID NO: 2

(B) a deletion of Glu at the 543^{rd} position and Asp at the 544^{th} position in an amino-acid sequence of SEQ ID NO: 2

(C) a mutation from Pro at the 502^{nd} position to Arg in an amino-acid sequence of SEQ ID NO: 5

The detection of the mutation may be conducted with respect to any one of them, two or more of them, or all of them. Further, the mutations of the (A) and (B) are preferably detected together.

A mutation of a polypeptide can be detected, by an antigen-antibody reaction between the antibody and a polypeptide in the sample, with an antibody, whose antigen is a mutant polypeptide of the present invention, for example. In this instance, when the antigen-antibody reaction between the antibody and a polypeptide in the sample is confirmed, it can be judged as having a mutation, and when the antigen-antibody reaction is not confirmed, it can be judged as not having a mutation. The type of an antibody is not particularly limited, and a monoclonal antibody, a polyclonal antibody, and the like can be used. The technique of the antigen-antibody reaction is not limited, and immunoblotting technique and immunohistochemical technique, and the like can be used.

### <CMPD Evaluation Kit>

A CMPD evaluation kit of the present invention includes, for example, a first evaluation kit that can be used for detecting the mutant nucleic acid of the present invention, and a second evaluation kit which can be used for detecting the mutant polypeptide of the present invention.

The first evaluation kit of the present invention, as described above, is an evaluation kit for evaluating CMPD and includes at least one of a probe that is a polynucleotide including at least one mutation selected from the group consisting of the (a), (b), and (c), or a polynucleotide composed of a base sequence complementary thereto, and that can hybridize specifically thereto, and a primer which is a polynucleotide including at least one mutation selected from the group consisting of the (a), (b), and (c), or a polynucleotide composed of the base sequence complementary thereto, and that can hybridize thereto. Further, the second evaluation kit of the present invention, as described above, includes an antibody, and an antigen thereof is the mutant polypeptide of the present invention. By using these evaluation kits of the present invention, the CMPD evaluation method of the present invention can be conducted simply and easily. In addition, a primer, a probe and, an antibody are similar to as those described above.

The configuration of the first evaluation kit of the present invention is not particularly limited and, for example, further may include a primer, DNA polymerase, dNTPs, various additives, which are required for the nucleic acid amplification. Further, the configuration of the second evaluation kit of the present invention is also not particularly limited and, for example, further may include a secondary antibody for detecting the antibody, and enzyme and substrate for detecting a label added to an antibody. In addition the evaluation kit of the present invention may further include an instruction manual.

### EXAMPLES

Next, examples of the present invention are described. However, the present invention is not limited by the following examples.

### Example 1

Genomic DNAs were prepared from total of 130 patients, PV patients (n=52), ET patients (n=55), IMF patients (n=3), CMP D-unclassified (CMPD-U) patents (n=15), and hyper eosinophilic syndrome (HES) patients (n=5) who are diagnosed by the heretofore known standard. Also genomic DNAs were prepared from healthy subjects. The genomic DNAs of the patients were extracted from buffy coats of bone marrow obtained from the patients and the genomic DNAs of the healthy subjects were extracted from peripheral blood granulocytes obtained from the healthy subjects, respectively, by using QIAamp DNA Mini Kit (trade name, manufactured by QIAGEN GmbH). The genomic DNAs of the healthy subjects were used as a normal control, and analysis of mutations and polymorphisms was conducted by using a mutation analysis apparatus (manufactured by ARKRAY, Inc.).

With respect to the genomic DNAs of 130 patients, for analyzing the mutation of JAK2^{V617F}, an ASP-PCR method and a Direct Sequencing method were conducted in accordance with a common procedure (Baxter E J, et al. Acquired mutation of the tyrosine kinase JAK2 in human myeloproliferative disorders. Lancet. 2005; 365: 1054-1061). Further, PCR products were introduced to a pCR (registered trademark) 2.1-TOPO vector by using a TOPO TA cloning kit (trade name, manufactured by Invitrogen Corporation) and a sequence of the PCR products were confirmed by a sequence of the resultant recombinant plasmid.

By the analysis, JAK2^{V617F} was confirmed in 31 out of 52 PV patients (59.6%), 28 out of 55 ET patients (50.9%), 1 out of 3 IMF patients (33.3%), 5 out of 15 CMPD-U patients (33.3%), and 1 out of 5 HES patients (20%). From the result, as described above, it was found that the onset rate of JAK2^{V617F}-positive PV patients of Japanese (59.6%) is lower than that of Occidental (65˜99%).

Next, with respect to genomic DNAs of the JAK2^{V617F}-negative CMPD patients, analysis of a mutation was conducted. As a result, the mutation (JAK2^{E543-D544 del}) in which G was substituted for A at a nucleotide residue at the 2116^{th} position of SEQ ID NO: 1 (at the 1621^{st} position in CDS of SEQ ID NO: 3) (JAK2^{R541K}), and a nucleotide residue (total of 6 bp) at the 2121^{st} to 2126^{th} position of SEQ ID NO: 1 (at the 1625^{th} to 1630^{th} position in CDS of SEQ ID NO: 3) was deleted, was found from JAK2^{V617F}-negative PV patients.

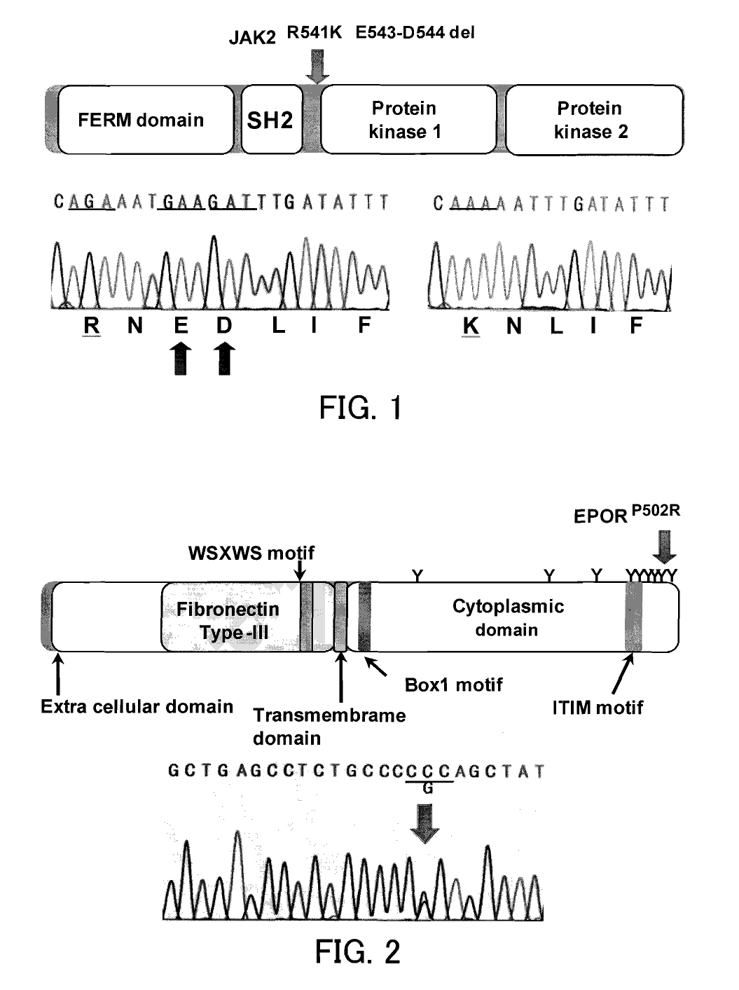

FIG. 1 shows a schematic diagram of a partial region of a JAK2 gene and a sequence result. In FIG. 1, arrows indicate positions of the mutation (JAK2^{R541K E543-D544 del}) which are newly found. In FIG. 1, the left-sided base sequence is a sequence of wild-type allele, and the right-sided base sequence is a sequence of variant allele. In addition, the underlined codons are codons in which mutation occurs. As shown in FIG. 1, JAK2^{R541K} and JAK2^{E543-D544 del} are new mutations existing between SH2 domain and Protein kinase 1 domain in the exon 12 (when considering exon 1 and exon 2 of a nontranscribed region, it is in the exon 14) of a JAK2 gene. This mutation was adjacent to the deletion mutation that already has been reported (Scott L M, et al. JAK2 exon 12 mutations in polycythemia vera and idiopathic erythrocytosis. N Engl J. Med. 2007; 356: 459-468.). In addition, with respect to an amino-acid residue at the 541^{st} position of SEQ ID NO: 2, arginine (R) is substituted for lysine (K) when G at the 2116^{th} position of SEQ ID NO: 1 is substituted for A. These mutations, JAK2^{R541K} and JAK2^{E543-D544 del}, were not detected from the healthy subjects.

With respect to one patient whom a mutation was detected, clinical characteristics are shown in the following Table 1. As a result, as shown in the following Table 1, it was found that although this patient was a PV patient of JAK2^{V617F}-negative, the typical clinical characteristics of PV were confirmed in the same manner as the patients of the JAK2^{V617F}-positive.

TABLE 1

| | | | | | | | | | | cytogenetical | JAK2V617F |
|---|---|---|---|---|---|---|---|---|---|---|---|
| | patient | age | gender | WBC | RBC | Hb | Ht | PLT | diagnosis | analysis | mutation |
| JAK2^{R541KE543-D544 del} | 1 | 52 | F | 6900 | 857 | 19 | 59.5 | 43 | PV | 46, XX | (—) |
| EPOR^{P478R} | 2 | 75 | M | 15700 | 276 | 4.9 | 19.8 | 525 | ET | 46, XX | (—) |
| | | | | | | | | | | del(11)(q21q23) | (—) |
| | 3 | 39 | F | 6900 | 432 | 13 | 38 | 103 | ET | 46, XX | (—) |

WBC: white blood cell counts (/μl)
RBC: red blood cell counts (×10⁴/μl)
Hb: hemoglobin concentration (mg/100 ml)
Ht: hematocrit value (%)
PLT: platelet counts (×10⁴/μl)

Next, with respect to the CMPD patients, the entire sequence of an EPOR gene was analyzed. As a result, the mutation (EPOR^{P502R}) in which C was substituted for G at a nucleotide residue at the 1641^{st} position of SEQ ID NO: 3. (at the 1505^{th} position in CDS of SEQ ID NO: 6) was confirmed from 2 ET patients who were JAK2^{V617F}-negative.

FIG. 2 shows a schematic diagram of a partial region of an EPOR gene and a sequence result. The thick arrows indicate the position of the newly found mutation (EPOR^{P478K}) and Y shows position of a tyrosine residue at a gene. The base sequence in FIG. 2 is a sequence of wild-type allele, underlined codons are codons in which mutation occurs and CCC mutates to CGC. As shown in FIG. 2, with respect to EPOR^{P502R}, proline is substituted for arginine. This mutation is a new mutation existing in the exon 8 which is a negative adjustment region of cytoplasmic domain of an EPOR gene. In addition, with respect to an amino-acid residue at the 502^{nd} position of SEQ ID NO: 4, proline is substituted for arginine when C at the 1641^{st} position of SEQ ID NO: 3 is substituted for G. This mutation, EPOR^{P502R}, was not detected from the healthy subjects.

As described above, because JAK2^{R541K}, JAK2^{E543-D544 del}, and EPOR^{P502R} were found from CMPD patients of JAK2^{V617F}-negative, the evaluation of the possibility of the onset of CMPD by detection of these mutations with respect to a specimen of AK2^{V617F}-negative which could not be evaluated, can be achieved. In addition, because these mutations were found from CMPD patients of JAK2^{V617F}-negative, these mutations are considered as not corresponding to a secondary mutation.

### INDUSTRIAL APPLICABILITY

As described above, because the mutant nucleic acid of the present invention and the mutant polypeptide, the expression product of the mutant nucleic acid of the present invention, are related to CMPD, they can be used as a marker of CMPD. Further, by detecting them, evaluation of the possibility of the onset of CMPD can be achieved. Particularly, because these mutations were detected from conventionally reported CMPD patients of JAK2^{V617F}-negative, by detecting the mutant nucleic acid and the mutant polypeptide of the present invention, evaluation of the possibility of the onset of CMPD could be achieved with respect to JAK2^{V617F}-negative patients who could not be evaluated by detection of JAK2^{V617F}. Therefore, the present invention is very useful technology in a clinical medical field, and the like. In addition, the mutation of the present invention and the relation between the mutations and diseases are found by the inventors for the first time.

1. A mutant nucleic acid composed of at least one nucleic acid selected from the group consisting of the following (i), (ii), and (iii):
(i) a nucleic acid composed of a JAK2 gene including any one of the following mutations (a) and (b) or a fragment thereof including the mutation
(a) a mutation from G at the 2116^{th} position to A in a base sequence of SEQ ID NO: 1
(b) a deletion of a nucleotide residue from the 2121^{st} to 2126^{th} position in a base sequence of SEQ ID NO: 1
(ii) a nucleic acid composed of an EPOR gene including the following mutation (c) or a fragment thereof including the mutation
(c) a mutation from C at the 1641^{st} position to G in a base sequence of SEQ ID NO: 4.
(iii) a nucleic acid composed of a base sequence complementary to at least one of the nucleic acid of (i) and the nucleic acid of (ii).

2. A mutant polypeptide composed of any one of the following polypeptides (I) and (II):
(I) a polypeptide composed of JAK2 protein including any one of the following mutations (A) and (B) or a fragment thereof including the mutation
(A) a mutation from Arg at the 540 position to Lys in an amino-acid sequence of SEQ ID NO: 2
(B) a deletion of Glu at the 543^{rd} position and Asp at the 544^{th} position in an amino-acid sequence of SEQ ID NO: 2
(II) a polypeptide composed of EPOR protein including the following mutation (C) or a fragment thereof including the mutation
(C) a mutation from Pro at the 502″ position to Arg in an amino-acid sequence of SEQ ID NO: 5.

3. A marker of CMPD including a mutant nucleic acid according to claim 1.

4. A marker of CMPD including a mutant polypeptide according to claim 2.

5. An evaluation method for evaluating the possibility of CMPD by detecting at least one mutation of a nucleic acid and a polypeptide in a biological sample, wherein
the evaluation method includes at least one process of the following (X) and (Y):
(X) a process for detecting the presence or absence of a mutant nucleic acid according to claim 1 in the sample
(Y) a process for detecting the presence or absence of a mutant polypeptide composed of any one of the following polypeptides (I) and (II):
(I) a polypeptide composed of JAK2 protein including any one of the following mutations (A) and (B) or a fragment thereof including the mutation
(A) a mutation from Arg at the 541^{st} position to Lys in an amino-acid sequence of SEQ ID NO: 2
(B) a deletion of Glu at the 543^{rd} position and Asp at the 544^{th} position in an amino-acid sequence of SEQ ID NO: 2
(II) a polypeptide composed of EPOR protein including the following mutation (C) or a fragment thereof including the mutation
(C) a mutation from Pro at the 502^{nd} position to Arg in an amino-acid sequence of SEQ ID NO: 5; in the sample.

6. The evaluation method according to claim 5, wherein, in the process (X), with respect to the nucleic acid in the sample, at least one mutation selected from the group consisting of the following (a), (b), and (c) is detected
(a) a mutation from G at the 2116^{th} position to A in a base sequence of SEQ ID NO: 1
(b) a deletion of a nucleotide residue from the 2121^{st} to 2126^{th} position in a base sequence of SEQ ID NO: 1
(c) a mutation from C at the 1641^{st} position to G in a base sequence of SEQ ID NO: 4.

7. The evaluation method according to claim 5, wherein, in the process (X), the mutation is detected with a probe which is a polynucleotide including at least one mutation selected from the group consisting of the (a), (b), and (c) or a polynucleotide composed of a base sequence complementary thereto, and that can hybridize to a mutant nucleic acid composed of at least one nucleic acid selected from the group consisting of the following (i), (ii), and (iii);
(i) a nucleic acid composed of a JAK2 gene including any one of the following mutations (a) and (b) or a fragment thereof including the mutation
(a) a mutation from G at the 2116^{th} position to A in a base sequence of SEQ ID NO: 1
(b) a deletion of a nucleotide residue from the 2121^{st} to 2126^{th} position in a base sequence of SEQ ID NO: 1
(ii) a nucleic acid composed of an EPOR gene including the following mutation (c) or a fragment thereof including the mutation
(c) a mutation from C at the 1641^{st} position to G in a base sequence of SEQ ID NO: 4.
(iii) a nucleic acid composed of a base sequence complementary to at least one of the nucleic acid of (i) and the nucleic acid of (ii),
by hybridization of the probe and a nucleic acid of the sample.

8. The evaluation method according to claim 5, wherein, in the process (X), the mutation is detected with a primer that is a polynucleotide including at least one mutation selected from the group consisting of the (a), (b), and (c) or a polynucleotide composed of a base sequence complementary thereto, and that can hybridize to a mutant nucleic acid composed of at least one nucleic acid selected from the group consisting of the following (i), (ii), and (iii):
(i) a nucleic acid composed of a JAK2 gene including any one of the following mutations (a) and (b) or a fragment thereof including the mutation
(a) a mutation from G at the 2116^{th} position to A in a base sequence of SEQ ID NO: 1
(b) a deletion of a nucleotide residue from the 2121^{st} to 2126^{th} position in a base sequence of SEQ ID NO: 1
(ii) a nucleic acid composed of an EPOR gene including the following mutation (c) or a fragment thereof including the mutation
(c) a mutation from C at the 1641^{st} position to G in a base sequence of SEQ ID NO: 4.
(iii) a nucleic acid composed of a base sequence complementary to at least one of the nucleic acid of (i) and the nucleic acid of (ii),
by a nucleic acid amplification reaction in which a nucleic acid in the sample is used as a template.

9. The evaluation method according to claim 8, wherein the primer has the mutant base or a base complementary to the mutant base at 3′ region.

10. The evaluation method according to claim 5, wherein, in the process (Y), with respect to the polypeptide in the sample, at least one mutation selected from the group consisting of the following (A), (B), and (C) is detected
(A) a mutation from Arg at the 541^{st} position to Lys in an amino-acid sequence of SEQ ID NO: 2
(B) a deletion of Glu at the 543^{rd} position and Asp at the 544^{th} position in an amino-acid sequence of SEQ ID NO: 2
(C) a mutation from Pro at the 502^{nd} position to Arg in an amino-acid sequence of SEQ ID NO: 5.

11. The evaluation method according to claim 5, wherein, in the process (Y), the mutation is detected with an antibody, an antigen thereof is a mutant polypeptide composed of any one of the following polypeptides (I) and (II):
(I) a polypeptide composed of JAK2 protein including any one of the following mutations (A) and (B) or a fragment thereof including the mutation
(A) a mutation from Arg at the 541^{st} position to Lys in an amino-acid sequence of SEQ ID NO: 2
(B) a deletion of Glu at the 543^{rd} position and Asp at the 544^{th} position in an amino-acid sequence of SEQ ID NO: 2
(II) a polypeptide composed of EPOR protein including the following mutation (C) or a fragment thereof including the mutation
(C) a mutation from Pro at the 502^{nd} position to Arg in an amino-acid sequence of SEQ ID NO: 5,
by an antigen-antibody reaction of the antibody and the polypeptide in the sample.

12. The evaluation method according to claim 5, wherein the sample is a biological sample in which a JAK2^{V617F} mutation is negative.

13. An evaluation kit for evaluating CMPD, wherein the evaluation kit includes any one of
a probe which is a polynucleotide including at least one mutation selected from the group consisting of the following (a), (b), and (c) or a polynucleotide composed of a base sequence complementary thereto, and can hybridize to a mutant nucleic acid according to claim 1 and
a primer which is a polynucleotide including at least one mutation selected from the group consisting of the following (a), (b), and (c)
(a) a mutation from G at the 2116^{th} position to A in a base sequence of SEQ ID NO: 1
(b) a deletion of a nucleotide residue from the 2121^{st} to 2126^{th} position in a base sequence of SEQ ID NO: 1
(c) a mutation from C at the 1641^{st} position to G in a base sequence of SEQ ID NO: 4.
or a polynucleotide composed of a base sequence complementary thereto, and can hybridize to the mutant nucleic acid according to claim 1.

14. An evaluation kit for evaluating CMPD, wherein the evaluation kit includes an antibody, and an antigen thereof is a mutant polypeptide according to claim 2.


SEQUENCE LISTING (6 sequences)

SEQ ID NO: 1 - DNA, 5097 bp, Homo sapiens
  CDS (495)..(3893)
ctgcaggaaggagagaggaagaggagcagaagggggcagcagcggacgccgctaacggcctccctcggcgctgacaggctgggccggcgcccggctcgcttgggtgttcgcgtcgccacttcggcttctcggccggtcgggcccctcggcccgggcttgcggcgcgcgtcggggctgagggctgctgcggcgcagggagaggcctggtcctcgctgccgagggatgtgagtgggagctgagcccacactggagggcccccgagggcccagcctggaggtcgttcagagccgtgcccgccccggggcttcgcagaccttgacccgccgggtaggagccgcccctgcgggctcgagggcgcgctctggtcgcccgatctgtgtagccggtttcagaagcaggcaacaggaacaagatgtgaactgtttctcttctgcagaaaaagaggctcttcctcctcctcccgcgacggcaaatgttctgaaaaagactctgcatgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctagggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatgaaagaaatgaccttcattctgagaccaaagtagatttacagaacaaagttttatatttcacattgctgtggactattattacatatatcattattatataaatcatgatgctagccagcaaagatgtgaaaatatctgctcaaaactttcaaagtttagtaagtttttcttcatgaggccaccagtaaaagacattaatgagaattccttagcaaggattttgtaagaagtttcttaaacattgtctgttaacatcactcttgtctggcaaaagaaaaaaaatagactttttcaactcagctttttgagacctgaaaaaattattatgtaaattttgcaatgttaaagatgcacagaatatgtatgtatagtttttaccacagtggatgtataataccttggcatcttgtgtgatgttttacacacatgagggctggtgttcattaatactgttttctaatttttccatagttaatctataattaattacttcactatacaaacaaattaagatgttcagataattgaataagtacctttgtgtccttgttcatttatatcgctggccagcattataagcaggtgtatacttttagcttgtagttccatgtactgtaaatatttttcacataaagggaacaaatgtctagttttatttgtataggaaatttccctgaccctaaataatacattttgaaatgaaacaagcttacaaagatataatctattttattatggtttcccttgtatctatttgtggtgaatgtgttttttaaatggaactatctccaaatttttctaagactactatgaacagttttcttttaaaattttgagattaagaatgccaggaatattgtcatcctttgagctgctgactgccaataacattcttcgatctctgggatttatgctcatgaactaaatttaagcttaagccataaaatagattagattgttttttaaaaatggatagctcattaagaagtgcagcaggttaagaattttttcctaaagactgtatatttgaggggtttcagaattttgcattgcagtcatagaagagatttatttcctttttagaggggaaatgaggtaaataagtaaaaaagtatgcttgttaattttattcaagaatgccagtagaaaattcataacgtgtatctttaagaaaaatgagcatacatcttaaatcttttcaatta

SEQ ID NO: 2 - protein, 1132 aa, Homo sapiens
MGMACLTMTEMEGTSTSSIYQNGDISGNANSMKQIDPVLQVYLYHSLGKSEADYLTFPSGEYVAEEICIAASKACGITPVYHNMFALMSETERIWYPPNHVFHIDESTRHNVLYRIRFYFPRWYCSGSNRAYRHGISRGAEAPLLDDFVMSYLFAQWRHDFVHGWIKVPVTHETQEECLGMAVLDMMRIAKENDQTPLAIYNSISYKTFLPKCIRAKIQDYHILTRKRIRYRFRRFIQQFSQCKATARNLKLKYLINLETLQSAFYTEKFEVKEPGSGPSGEEIFATIIITGNGGIQWSRGKHKESETLTEQDLQLYCDFPNIIDVSIKQANQEGSNESRVVTIHKQDGKNLEIELSSLREALSFVSLIDGYYRLTADAHHYLCKEVAPPAVLENIQSNCHGPISMDFAISKLKKAGNQTGLYVLRCSPKDFNKYFLTFAVERENVIEYKHCLITKNENEEYNLSGTKKNFSSLKDLLNCYQMETVRSDNIIFQFTKCCPPKPKDKSNLLVFRTNGVSDVPTSPTLQRPTHMNQMVFHKIRNEDLIFNESLGQGTFTKIFKGVRREVGDYGQLHETEVLLKVLDKAHRNYSESFFEAASMMSKLSHKHLVLNYGVCVCGDENILVQEFVKFGSLDTYLKKNKNCINILWKLEVAKQLAWAMHFLEENTLIHGNVCAKNILLIREEDRKTGNPPFIKLSDPGISITVLPKDILQERIPWVPPECIENPKNLNLATDKWSFGTTLWEICSGGDKPLSALDSQRKLQFYEDRHQLPAPKWAELANLINNCMDYEPDFRPSFRAIIRDLNSLFTPDYELLTENDMLPNMRIGALGFSGAFEDRDPTQFEERHLKFLQQLGKGNFGSVEMCRYDPLQDNTGEVVAVKKLQHSTEEHLRDFEREIEILKSLQHDNIVKYKGVCYSAGRRNLKLIMEYLPYGSLRDYLQKHKERIDHIKLLQYTSQICKGMEYLGTKRYIHRDLATRNILVENENRVKIGDFGLTKVLPQDKEYYKVKEPGESPIFWYAPESLTESKFSVASDVWSFGVVLYELFTYIEKSKSPPAEFMRMIGNDKQGQMIVFHLIELLKNNGRLPRPDGCPDEIYMIMTECWNNNVNQRPSFRDLALRVDQIRDNMAG

SEQ ID NO: 3 - DNA, 3399 bp, Homo sapiens
atgggaatggcctgccttacgatgacagaaatggagggaacatccacctcttctatatatcagaatggtgatatttctggaaatgccaattctatgaagcaaatagatccagttcttcaggtgtatctttaccattcccttgggaaatctgaggcagattatctgacctttccatctggggagtatgttgcagaagaaatctgtattgctgcttctaaagcttgtggtatcacacctgtgtatcataatatgtttgctttaatgagtgaaacagaaaggatctggtatccacccaaccatgtcttccatatagatgagtcaaccaggcataatgtactctacagaataagattttactttcctcgttggtattgcagtggcagcaacagagcctatcggcatggaatatctcgaggtgctgaagctcctcttcttgatgactttgtcatgtcttacctctttgctcagtggcggcatgattttgtgcacggatggataaaagtacctgtgactcatgaaacacaggaagaatgtcttgggatggcagtgttagatatgatgagaatagccaaagaaaacgatcaaaccccactggccatctataactctatcagctacaagacattcttaccaaaatgtattcgagcaaagatccaagactatcatattttgacaaggaagcgaataaggtacagatttcgcagatttattcagcaattcagccaatgcaaagccactgccagaaacttgaaacttaagtatcttataaatctggaaactctgcagtctgccttctacacagagaaatttgaagtaaaagaacctggaagtggtccttcaggtgaggagatttttgcaaccattataataactggaaacggtggaattcagtggtcaagagggaaacataaagaaagtgagacactgacagaacaggatttacagttatattgcgattttcctaatattattgatgtcagtattaagcaagcaaaccaagagggttcaaatgaaagccgagttgtaactatccataagcaagatggtaaaaatctggaaattgaacttagctcattaagggaagctttgtctttcgtgtcattaattgatggatattatagattaactgcagatgcacatcattacctctgtaaagaagtagcacctccagccgtgcttgaaaatatacaaagcaactgtcatggcccaatttcgatggattttgccattagtaaactgaagaaagcaggtaatcagactggactgtatgtacttcgatgcagtcctaaggactttaataaatattttttgacttttgctgtcgagcgagaaaatgtcattgaatataaacactgtttgattacaaaaaatgagaatgaagagtacaacctcagtgggacaaagaagaacttcagcagtcttaaagatcttttgaattgttaccagatggaaactgttcgctcagacaatataattttccagtttactaaatgctgtcccccaaagccaaaagataaatcaaaccttctagtcttcagaacgaatggtgtttctgatgtaccaacctcaccaacattacagaggcctactcatatgaaccaaatggtgtttcacaaaatcagaaatgaagatttgatatttaatgaaagccttggccaaggcacttttacaaagatttttaaaggcgtacgaagagaagtaggagactacggtcaactgcatgaaacagaagttcttttaaaagttctggataaagcacacagaaactattcagagtctttctttgaagcagcaagtatgatgagcaagctttctcacaagcatttggttttaaattatggagtatgtgtctgtggagacgagaatattctggttcaggagtttgtaaaatttggatcactagatacatatctgaaaaagaataaaaattgtataaatatattatggaaacttgaagttgctaaacagttggcatgggccatgcattttctagaagaaaacacccttattcatgggaatgtatgtgccaaaaatattctgcttatcagagaagaagacaggaagacaggaaatcctcctttcatcaaacttagtgatcctggcattagtattacagttttgccaaaggacattcttcaggagagaataccatgggtaccacctgaatgcattgaaaatcctaaaaatttaaatttggcaacagacaaatggagttttggtaccactttgtgggaaatctgcagtggaggagataaacctctaagtgctctggattctcaaagaaagctacaattttatgaagataggcatcagcttcctgcaccaaagtgggcagaattagcaaaccttataaataattgtatggattatgaaccagatttcaggccttctttcagagccatcatacgagatcttaacagtttgtttactccagattatgaactattaacagaaaatgacatgttaccaaatatgaggataggtgccctagggttttctggtgcctttgaagaccgggatcctacacagtttgaagagagacatttgaaatttctacagcaacttggcaagggtaattttgggagtgtggagatgtgccggtatgaccctctacaggacaacactggggaggtggtcgctgtaaaaaagcttcagcatagtactgaagagcacctaagagactttgaaagggaaattgaaatcctgaaatccctacagcatgacaacattgtaaagtacaagggagtgtgctacagtgctggtcggcgtaatctaaaattaattatggaatatttaccatatggaagtttacgagactatcttcaaaaacataaagaacggatagatcacataaaacttctgcagtacacatctcagatatgcaagggtatggagtatcttggtacaaaaaggtatatccacagggatctggcaacgagaaatatattggtggagaacgagaacagagttaaaattggagattttgggttaaccaaagtcttgccacaagacaaagaatactataaagtaaaagaacctggtgaaagtcccatattctggtatgctccagaatcactgacagagagcaagttttctgtggcctcagatgtttggagctttggagtggttctgtatgaacttttcacatacattgagaagagtaaaagtccaccagcggaatttatgcgtatgattggcaatgacaaacaaggacagatgatcgtgttccatttgatagaacttttgaagaataatggaagattaccaagaccagatggatgcccagatgagatctatatgatcatgacagaatgctggaacaataatgtaaatcaacgcccctcctttagggatctagctcttcgagtggatcaaataagggataacatggctggatga

SEQ ID NO: 4 - DNA, 1849 bp, Homo sapiens
  CDS (137)..(1663)
acttagaggcgcctggtcgggaagggcctggtcagctgcgtccggcggaggcagctgctgacccagctgtggactgtgccgggggcgggggacggaggggcaggagccctgggctccccgtggcgggggctgtatcatggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttaggacaccaggctgcagatgatcagggatccaatatgactcagagaaccagtgcagactcaagacttatggaacagggatggcgaggcctctctcaggagcaggggcattgctgattttgtctgcccaatccatcctgctcaggaaaccacaaccttgcagtatttttaaatatgtatagtttttttg

SEQ ID NO: 5 - protein, 508 aa, Homo sapiens
MDHLGASLWPQVGSLCLLLAGAAWAPPPNLPDPKFESKAALLAARGPEELLCFTERLEDLVCFWEEAASAGVGPGNYSFSYQLEDEPWKLCRLHQAPTARGAVRFWCSLPTADTSSFVPLELRVTAASGAPRYHRVIHINEVVLLDAPVGLVARLADESGHVVLRWLPPPETPMTSHIRYEVDVSAGNGAGSVQRVEILEGRTECVLSNLRGRTRYTFAVRARMAEPSFGGFWSAWSEPVSLLTPSDLDPLILTLSLILVVILVLLTVLALLSHRRALKQKIWPGIPSPESEFEGLFTTHKGNFQLWLYQNDGCLWWSPCTPFTEDPPASLEVLSERCWGTMQAVEPGTDDEGPLLEPVGSEHAQDTYLVLDKWLLPRNPPSEDLPGPGGSVDIVAMDEGSEASSCSSALASKPSPEGASAASFEYTILDPSSQLLRPWTLCPELPPTPPHLKYLYLVVSDSGISTDYSSGDSQGAQGGLSDGPYSNPYENSLIPAAEPLPPSYVACS

SEQ ID NO: 6 - DNA, 1527 bp, Homo sapiens
atggaccacctcggggcgtccctctggccccaggtcggctccctttgtctcctgctcgctggggccgcctgggcgcccccgcctaacctcccggaccccaagttcgagagcaaagcggccttgctggcggcccgggggcccgaagagcttctgtgcttcaccgagcggttggaggacttggtgtgtttctgggaggaagcggcgagcgctggggtgggcccgggcaactacagcttctcctaccagctcgaggatgagccatggaagctgtgtcgcctgcaccaggctcccacggctcgtggtgcggtgcgcttctggtgttcgctgcctacagccgacacgtcgagcttcgtgcccctagagttgcgcgtcacagcagcctccggcgctccgcgatatcaccgtgtcatccacatcaatgaagtagtgctcctagacgcccccgtggggctggtggcgcggttggctgacgagagcggccacgtagtgttgcgctggctcccgccgcctgagacacccatgacgtctcacatccgctacgaggtggacgtctcggccggcaacggcgcagggagcgtacagagggtggagatcctggagggccgcaccgagtgtgtgctgagcaacctgcggggccggacgcgctacaccttcgccgtccgcgcgcgtatggctgagccgagcttcggcggcttctggagcgcctggtcggagcctgtgtcgctgctgacgcctagcgacctggaccccctcatcctgacgctctccctcatcctcgtggtcatcctggtgctgctgaccgtgctcgcgctgctctcccaccgccgggctctgaagcagaagatctggcctggcatcccgagcccagagagcgagtttgaaggcctcttcaccacccacaagggtaacttccagctgtggctgtaccagaatgatggctgcctgtggtggagcccctgcacccccttcacggaggacccacctgcttccctggaagtcctctcagagcgctgctgggggacgatgcaggcagtggagccggggacagatgatgagggccccctgctggagccagtgggcagtgagcatgcccaggatacctatctggtgctggacaaatggttgctgccccggaacccgcccagtgaggacctcccagggcctggtggcagtgtggacatagtggccatggatgaaggctcagaagcatcctcctgctcatctgctttggcctcgaagcccagcccagagggagcctctgctgccagctttgagtacactatcctggaccccagctcccagctcttgcgtccatggacactgtgccctgagctgccccctaccccaccccacctaaagtacctgtaccttgtggtatctgactctggcatctcaactgactacagctcaggggactcccagggagcccaagggggcttatccgatggcccctactccaacccttatgagaacagccttatcccagccgctgagcctctgccccccagctatgtggcttgctcttag